(19) United States
(12) Patent Application Publication
(10) Pub. No.: US 2017/0281982 A1
(43) Pub. Date: Oct. 5, 2017

(54) METHODS AND SYSTEMS FOR GENERATING AN OCCLUSION USING ULTRASOUND

(71) Applicant: General Electric Company, Schenectady, NY (US)

(72) Inventors: ShangXian Zhu, Wauwatosa, WI (US); Bruno Hans Haider, Niskayuna, NY (US); Lavanya Kiran, Bangalore (IN); Edward Leahy, Weymouth, MA (US); Milki Tilimo, Everett, MA (US); Heather Linaya Vahdat, Apex, NC (US)

(73) Assignee: Family Health International, Durham, NC (US)

(21) Appl. No.: 15/087,624

(22) Filed: Mar. 31, 2016

(51) Int. Cl. A61N 7/00

(52) U.S. Cl. CPC A61N 7/00; A61N 2007/0082; A61N 2007/0052; A61N 2007/0043

(57) ABSTRACT

An intra-cavity ultrasound imaging and therapy system is provided. The system includes an intra-cavity ultrasound probe including a housing configure to be inserted into a cavity proximate to a region of interest (ROI). The housing includes a transducer array located proximate to a distal end of the housing. The system also includes a diagnostic control circuit configured to direct the transducer array to collect diagnostic ultrasound signals from the ROI. The diagnostic control circuit is configured to generate an ultrasound image based on the diagnostic ultrasound signals. The diagnostic control circuit is further configured to direct the transducer array to deliver a high intensity focused ultrasound (HIFU) therapy at a treatment location based on target information derived from the ultrasound image.

### FIELD

Embodiments described herein generally relate to generating one or more occlusions using ultrasound signals generated by an ultrasound probe.

### BACKGROUND OF THE INVENTION

Permanent contraceptive methods among women in developing countries are limited by geographical, educational, and financial barriers. Healthcare providers are burdened with lack of infrastructure, access to basic hygiene and emergency necessities, a paucity of adequately trained staff, and breakdowns in the supply chain. Women seeking sterilization face their own set of barriers, including access to the procedure, lack of ability to pay, loss of work and wages, and time allotted for transportation and recovery. Although rare, complications arising from tubal sterilizations may be serious, involving infection or anesthetic complications. Additionally, conventional tubal sterilization methods may not be permanent requiring subsequent procedures, rely on hormonal treatments, use invasive surgical procedures, and unaffordable.

Conventional non-surgical approaches to achieve permanent contraception have focused primarily on techniques that require the instillation of chemical agents, such as quinacrine and polidocanol. However, such approaches are challenged by the ability to deliver a precise treatment and duration of the chemical agent, given the somewhat variable nature of fluid movement through the uterus and fallopian tubes.

For this and other reasons a new method and system is desired for a minimally invasive contraception for women.

### BRIEF DESCRIPTION OF THE INVENTION

In an embodiment a system (e.g., an intra-cavity ultrasound imaging and therapy system) is provided. The system includes an intra-cavity ultrasound probe including a housing configure to be inserted into a cavity proximate to a region of interest (ROI). The housing includes a transducer array located proximate to a distal end of the housing. The system also includes a diagnostic control circuit configured to direct the transducer array to collect diagnostic ultrasound signals from the ROI. The diagnostic control circuit is configured to generate an ultrasound image based on the diagnostic ultrasound signals. The diagnostic control circuit is further configured to direct the transducer array to deliver a high intensity focused ultrasound (HIFU) therapy at a treatment location based on target information derived from the ultrasound image.

In another embodiment a method (e.g., for generating an occlusion by delivering high intensity frequency ultrasound (HIFU) therapy) is provided. The method includes positioning an intra-cavity ultrasound probe into a cavity proximate to a region of interest (ROI). The intra-cavity ultrasound probe includes a housing. The housing includes a transducer array located at a distal end of the housing. The method further collecting diagnostic ultrasound signals at the transducer array from the ROI, and identifying a treatment location based on the diagnostic ultrasound signals. The method further includes delivering high intensity frequency ultrasound (HIFU) therapy from the transducer array to the treatment location.

In another embodiment a system (e.g., an intra-cavity ultrasound imaging and therapy system) is provided. The system includes an intra-cavity ultrasound probe including a housing configure to be inserted into a cavity proximate to a region of interest (ROI). The housing includes a transducer array located at a distal end of the housing. The system also includes a diagnostic control circuit configured to direct the transducer array to collect diagnostic ultrasound signals from the ROI. The diagnostic control circuit configured to generate an ultrasound image based on the diagnostic ultrasound signals. The system also includes a display to display the ultrasound image, and a user interface to receive a user input indicative of the treatment location. The diagnostic control circuit is configured to direct the transducer array to deliver a high intensity focused ultrasound (HIFU) therapy at the treatment location.

### BRIEF DESCRIPTION OF THE DRAWINGS

FIG. 1 illustrates a schematic block diagram of an ultrasound imaging system, in accordance with an embodiment.

FIG. 2 is an illustration of a simplified block diagram of a controller circuit of the ultrasound imaging system of FIG. 1, in accordance with an embodiment.

FIG. 3 illustrates a peripheral view of an ultrasound probe of the ultrasound imaging system, in accordance with an embodiment.

FIG. 4 illustrates a top view of the ultrasound probe shown in FIG. 3, in accordance with an embodiment.

FIG. 5 illustrates a transducer element of a transducer array, in accordance with an embodiment.

FIG. 6 illustrates various ultrasound probes, in accordance with various embodiments.

FIG. 7 illustrates a flowchart of a method for delivering high intensity focused ultrasound therapy at a treatment location, in accordance with an embodiment.

FIG. 8 illustrates an intra-cavity ultrasound probe positioned within a region of interest, in accordance with an embodiment.

FIG. 9 illustrates an ultrasound image shown on a display of the ultrasound imaging system shown in FIG. 1, in accordance with an embodiment.

FIG. 10 illustrates the intra-cavity ultrasound probe of FIG. 8 and the treatment location, in accordance with an embodiment.

FIG. 11 illustrates the intra-cavity ultrasound probe of FIG. 8 and the treatment location, in accordance with an embodiment.

FIG. 12 illustrates timing diagram of activation of transducer elements of a transducer array during a therapy session, in accordance with an embodiment.

### DETAILED DESCRIPTION OF THE INVENTION

The following detailed description of certain embodiments will be better understood when read in conjunction with the appended drawings. To the extent that the figures illustrate diagrams of the functional modules of various embodiments, the functional blocks are not necessarily indicative of the division between hardware circuitry. Thus, for example, one or more of the functional blocks (e.g., processors or memories) may be implemented in a single piece of hardware (e.g., a general purpose signal processor or a block of random access memory, hard disk, or the like). Similarly, the programs may be stand-alone programs, may be incorporated as subroutines in an operating system, may be functions in an installed software package, and the like. It should be understood that the various embodiments are not limited to the arrangements and instrumentality shown in the drawings.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising” or “having” an element or a plurality of elements having a particular property may include additional elements not having that property.

Various embodiments provide systems and methods for using ultrasound for a non-hormonal, non-surgical, non-implant contraception procedure. In operation, an intra-cavity ultrasound probe, such as a trans-vaginal ultrasound probe, may include a transducer array configured to deliver an imaging guidance and a high intensity focused ultrasound (HIFU) therapy to a treatment location within a cavity. Additionally or alternatively, the transducer array may be a plurality of separate transducer elements or segments. For example, a first transducer element of the transducer array may be configured for imaging guidance and a second transducer element of the transducer array may be configured to deliver the HIFU therapy.

During the imaging guidance, the intra-cavity ultrasound probe may be positioned within a cavity proximate to a region of interest (ROI) to create an ultrasound image of the ROI. For example, the ROI may correspond to fallopian tubes within a patient. Target information may be identified or selected based on the ultrasound image during the image guided therapy. For example, the target information may identify an anatomical target such as one of the uterotubal junctions or corneal junctions of the fallopian tubes. In operation, the anatomical target may be measured (e.g., length, diameter) to define a depth range, a sweep angle arc, and/or the like over which the HIFU therapy is delivered to the anatomical target.

The intra-cavity ultrasound probe may deliver the HIFU therapy to the anatomical target creating scar tissue, which can cause an occlusion within a tubular structure. For example, the HIFU therapy may form an occlusion at the uterotubal junctions or corneal junctions of the fallopian tube. The occlusion preventing the metaphase II-arrested oocyte (e.g., egg) from moving into the uterus and sperm from moving into the fallopian tube. Since fertilization normally occurs in the fallopian tube such occlusion formed by the HIFU therapy can prevent conception. Optionally, the imaging guidance and HIFU therapy may be repeated to other anatomical targets within the ROI (e.g., a second uterotubal junction or corneal junction of the fallopian tubes).

A technical effect of at least one embodiment described herein provides a non-hormonal, non-surgical, non-implant method of contraception. A technical effect of at least one embodiment described herein provides a non-invasive tubal occlusion to the fallopian tubes. A technical effect of at least one embodiment described herein eliminates the need for complex operating room infrastructure to perform a sterilization procedure. A technical effect of at least one embodiment described herein reduces a cost of sterilization procedures. A technical effect of at least one embodiment reduces the technical expertise of clinicians performing sterilization procedures (e.g., may be performed by midwives or physicians) and expanding access and availability of sterilization procedures. A technical effect of at least one embodiment reduces medical risk to the patient during sterilization procedures.

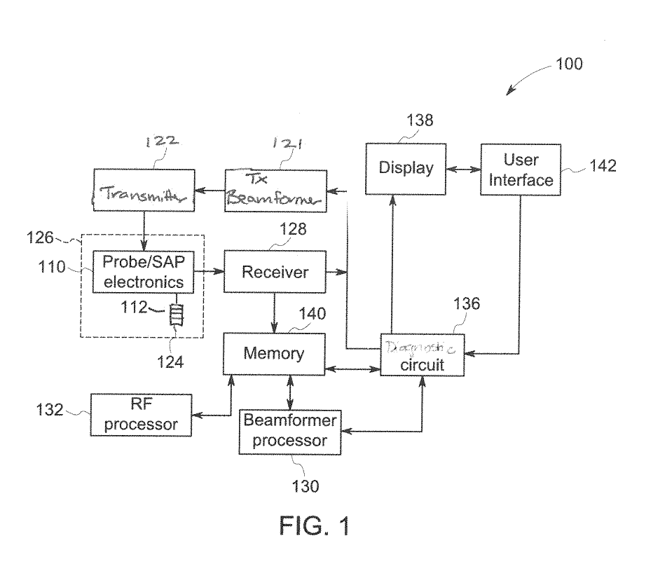

FIG. 1 is a schematic diagram of a diagnostic medical imaging system, specifically, an ultrasound imaging system 100. The ultrasound imaging system 100 includes an intra-cavity ultrasound probe 126 having probe/SAP electronics 110. The intra-cavity ultrasound probe 126 may be configured to acquire ultrasound data or information within a cavity (e.g., vaginal cavity, uterine cavity, ear canal, rectal cavity) proximate to and/or containing a region of interest (e.g., organ, blood vessel, fallopian tube(s)) of the patient for generating one or more ultrasound images. Additionally, the intra-cavity ultrasound probe 126 may be configured to transmit and/or deliver high intensity frequency ultrasound (HIFU) signals during the HIFU therapy to one or more treatment locations of the region of interest.

The intra-cavity ultrasound probe 126 is communicatively coupled to the diagnostic control circuit 136 via a transmitter 122. The transmitter 122 may transmit a signal to a transmit beamformer 121 based on acquisition settings received by the user and/or calculated by the diagnostic control circuit 136. Additionally, the transmitter 122 may transmit a signal to the transmit beamformer 121 based on HIFU parameters received by the user and/or calculated by the diagnostic control circuit 136. The signal transmitted by the transmitter 122 in turn drives the transducer elements 124 within the transducer array 112 during the imaging guidance and/or HIFU therapy. The transducer elements 124 emit pulsed ultrasonic signals into a patient (e.g., a body). The ultrasonic signals may include ultrasound imaging signals and/or HIFU signals delivered or emitted by the transducer elements 124. For example, the diagnostic control circuit 136 may direct the transducer array 112 to deliver HIFU therapy at a treatment location based on target information derived from an ultrasound image.

A variety of a geometries and configurations may be used for the array 112. Further, the array 112 of transducer elements 124 may be provided as part of, for example, different types of ultrasound probes. Optionally, the intra-cavity ultrasound probe 126 may include one or more tactile buttons (not shown). For example, a pressure sensitive tactile button may be positioned adjacent to the transducer array 122 of the intra-cavity ultrasound probe 126.

The acquisition settings may define an amplitude, pulse width, frequency, and/or the like of the ultrasound imaging signals emitted by the transducer elements 124. The acquisition settings may be adjusted by the user by selecting a gain setting, power, time gain compensation (TGC), resolution, and/or the like from the user interface 142. Additionally or alternatively, the acquisition settings may be algorithmically derived from one or more ultrasound images stored in the memory 140. For example, the diagnostic control circuit 136 may execute an algorithm stored in the memory 140 to adjust the TGC such that the uniformity of the one or more ultrasound images are increased.

The HIFU parameters may define a depth range, center frequency, amplitude or intensity, sweep angle arc, and/or the like over which the HIFU therapy is delivered by the transducer array 112 based on targeting information. For example, the centers frequency of the HIFU parameters may range from 0.5 MHz to 5 MHz. The intensity of the HIFU parameters may correspond to a power of the HIFU therapy delivered to the treatment location. For example, the power of the HIFU therapy may range from 300 to 3,000 mW/cm². It may be noted that the HIFU therapy having a power of less than 720 mW/cm² is preferable to remain within the power limits defined by the Federal Drug Administration of the United States. The depth range may define a distance from the transducer array 112 that will receive the HIFU therapy. The sweep angle arc may define a steering angle the transducer array 112 that will receive the HIFU therapy.

The HIFU parameters may be defined by the diagnostic control circuit 136 based on target information of the treatment location. The target information may include a distance, orientation, relative position, boundary locations and/or the like of the anatomical target relative to a reference position on the transducer array 112 or the intra-cavity ultrasound probe 126.

The transducer elements 124, for example piezoelectric crystals, emit the pulsed ultrasonic signals (e.g., ultrasound imaging signals, HIFU signals) into a body (e.g., patient) or volume corresponding to the acquisition settings and/or the HIFU parameters along one or more scan planes.

The ultrasound imaging signals of the ultrasonic signals may include, for example, one or more reference pulses, one or more pushing pulses (e.g., shear-waves), and/or one or more pulsed wave Doppler pulses. At least a portion of the ultrasound imaging signals back-scatter from a region of interest (ROI) (e.g., fallopian tubes, polyp within a rectum, and/or the like) to produce echoes. The echoes are delayed in time and/or frequency according to a depth, sweep angle, or movement, and are received by the transducer elements 124 within the transducer array 112. The ultrasound imaging signals may be used for imaging, for generating and/or tracking shear-waves, for measuring changes in position or velocity within the ROI, differences in compression displacement of the tissue (e.g., strain), and/or for therapy, among other uses.

The HIFU signals of the ultrasonic signals may be configured to have a higher focal intensity relative to the ultrasound imaging signals at the treatment location. For example, the HIFU signals may increase a temperature of the treatment location relative to areas of the anatomical target or ROI not receiving the HIFU signals. The increase in temperature caused by the HIFU signals may stimulate an inflammatory response at and/or around the treatment location, which may result in the production of scarring. Additionally or alternatively, the scarring may be used to form an occlusion within a tubular structure. For example, the HIFU therapy having a treatment location at the uterotubal junctions or corneal junctions of the fallopian tube can create an occlusion within the fallopian tube. In various embodiments, the transmitter 122 may receive the HIFU signals from the diagnostic control circuit 136.

The diagnostic control circuit 136 may be configured to direct one or more of the transducer elements 124 in the transducer array 122 transmitter 112 to deliver the HIFU therapy. Additionally or alternatively, the diagnostic control circuit 136 may define at least one of the HIFU parameters (e.g., depth range, sweep angle arc, electrical characteristics, and/or the like) over which the HIFU therapy is delivered based on the target information. For example, based on the target information, the diagnostic control circuit 136 may define one or more electrical characteristics corresponding to the HIFU parameters, which define the HIFU signals. For example, the diagnostic control circuit 136 may define an amplitude, a frequency, phase, and/or the like of the HIFU signals.

Optionally, the diagnostic control circuit 136 may be operably coupled to the transmit beamformer 121. For example, the transmit beamformer 121 may be configured to steer or to control the location and movement of a focal point of the HIFU signals based on instructions received by the diagnostic control circuit 136. In various embodiments, the transmit beamformer 121 may control electronic or mechanical steering of the intra-cavity ultrasound probe 126 to move and/or define the focal point to one or more of the treatment locations within and/or at the anatomical target based on the depth range, sweep angle arc, and/or the like determined by the diagnostic control circuit 136.

The diagnostic control circuit 136 may be operably coupled to a user interface 142. In various embodiments, the diagnostic control circuit 136 may be configured to determine, based on the target information, one or more HIFU parameters (e.g., depth range, sweep angle arc) relative to a reference position on the transducer array 112. In operation, the diagnostic control circuit 136 may be configured to perform one or more processing operations to determine target information, which may be used to identify the anatomical target and/or treatment location for the HIFU therapy

For example, the diagnostic control circuit 136 may receive a user input indicative of the treatment location from the user interface 124. In operation, the user input may represent a user designated points based on an ultrasound image indicative of at least a portion of a boundary of the anatomical target within the ROI. The diagnostic control circuit 136 may utilize the user input as the target information to designate or locate the treatment location within the ultrasound image. For example, the diagnostic control circuit 136 may calculate an overall size, shape, and/or position of the anatomical target relative to a reference position on the transducer array 112. The diagnostic control circuit 136 may determine the size and/or shape of the anatomical target by executing a segmentation algorithm stored on the memory 140. For example, when executing the segmentation algorithm, the diagnostic control circuit 136 may identify intensity changes and/or gradients of the vector data values, which form the ultrasound image to identify the size, shape, contour, and/or the like corresponding to the anatomical target.

In operation, based on the position of the anatomical target relative to the reference position on the transducer array 112, the size and shape of the anatomical target, and/or the like, the diagnostic control circuit 136 may determine the depth range and the sweeping angle width. The depth range and the sweeping angle width relates to the configuration or operation of the transducer elements 124 or probe 126 during the HIFU therapy. For example, the depth range and the sweeping angle width may define a focal point of the anatomical target corresponding to at least a portion of the treatment location for the HIFU signals during the HIFU therapy. The depth range may correspond to a distance range or bandwidth from the transducer array 112 within the focal point. For example, the depth range may define a range relative to the transducer array 112 corresponding to at least a portion of the treatment location over which the HIFU therapy is delivered. The sweep angle arc may correspond to an angle range relative to the transducer array 112 within the focal point. For example, the sweep angle arc may define a vertical range relative to the probe 126 correspond to at least a portion of the treatment location over which the HIFU therapy is delivered.

Optionally, the diagnostic control circuit 136 may determine additional HIFU parameters based on the target information. For example, the diagnostic control circuit 136 may determine HIFU parameters defining one or more electrical specifications (e.g., frequency, amplitude) of the HIFU signals.

The diagnostic control circuit 136 may include one or more processors. Optionally, the diagnostic control circuit 136 may include a central controller circuit (CPU), one or more microprocessors, or any other electronic component capable of processing inputted data according to specific logical instructions. Additionally or alternatively, the diagnostic control circuit 136 may execute instructions stored on a tangible and non-transitory computer readable medium (e.g., the memory 140) to perform one or more operations as described herein.

The transducer array 112 may have a variety of array geometries and configurations for the transducer elements 124 which may be provided as part of, for example, different types of ultrasound probes 126. The probe/SAP electronics 110 may be used to control the switching of the transducer elements 124. The probe/SAP electronics 110 may also be used to group the transducer elements 124 into one or more sub-apertures.

The diagnostic control circuit 136 may direct the transducer array 112 to collected diagnostic ultrasound signals from the ROI. For example, the transducer elements 124 may convert the received echo signals in response to the ultrasound imaging signals into electrical signals which may be received by a receiver 128. The receiver 128 may include one or more amplifiers, an analog to digital converter (ADC), and/or the like. The receiver 128 may be configured to amplify the received echo signals after proper gain compensation and convert these received analog signals from each transducer element 124 to diagnostic ultrasound signals sampled uniformly in time. The diagnostic ultrasound signals representing the received echoes are stored on memory 140, temporarily. The diagnostic ultrasound signals correspond to the backscattered waves receives by each transducer element 124 at various times. After digitization, the diagnostic ultrasound signals still may preserve the amplitude, frequency, phase information of the backscatter waves.

Optionally, the diagnostic control circuit 136 may retrieve the diagnostic ultrasound signals stored in the memory 140 to prepare for the beamformer processor 130. For example, the diagnostic control circuit 136 may convert the diagnostic ultrasound signals to baseband signals or compressing the diagnostic ultrasound signals.

The beamformer processor 130 may include one or more processors. Optionally, the beamformer processor 130 may include a central controller circuit (CPU), one or more microprocessors, or any other electronic component capable of processing inputted data according to specific logical instructions. Additionally or alternatively, the beamformer processor 130 may execute instructions stored on a tangible and non-transitory computer readable medium (e.g., the memory 140) for beamforming calculations using any suitable beamforming method such as adaptive beamforming, synthetic transmit focus, aberration correction, synthetic aperture, clutter reduction and/or adaptive noise control, and/or the like.

The beamformer processor 130 may further perform filtering and decimation, such that only the diagnostic ultrasound signals corresponding to relevant signal bandwidth is used, prior to beamforming of the diagnostic ultrasound signals. For example, the beamformer processor 130 may form packets of the diagnostic ultrasound signals based on scanning parameters corresponding to focal zones, expanding aperture, imaging mode (B-mode, color flow), and/or the like. The scanning parameters may define channels and time slots of the diagnostic ultrasound signals that may be beamformed, with the remaining channels or time slots of diagnostic ultrasound signals that may not be communicated for processing (e.g., discarded).

The beamformer processor 130 performs beamforming on the diagnostic ultrasound signals and outputs a radio frequency (RF) signal. The RF signal is then provided to an RF processor 132 that processes the RF signal. The RF processor 132 may generate different ultrasound image data types, e.g., B-mode, for multiple scan planes or different scanning patterns. The RF processor 132 gathers the information (e.g. I/Q, B-mode) related to multiple data slices and stores the data information, which may include time stamp and orientation/rotation information, in the memory 140.

Alternatively, the RF processor 132 may include a complex demodulator (not shown) that demodulates the RF signal to form IQ data pairs representative of the echo signals. The RF or IQ signal data may then be provided directly to the memory 140 for storage (e.g., temporary storage). Optionally, the output of the beamformer processor 130 may be passed directly to the diagnostic control circuit 136.

The diagnostic control circuit 136 may be configured to process the acquired ultrasound data (e.g., RF signal data, IQ data pairs, and/or the like). Optionally, the acquired ultrasound data may be processed by the diagnostic control circuit 136 during the imaging guidance as the echo signals are received. The diagnostic control circuit 136 may further create one or more ultrasound images based on the diagnostic ultrasound signals for display on the display 138. The diagnostic control circuit 136 may include one or more processors. Optionally, the diagnostic control circuit 136 may include a central controller circuit (CPU), one or more microprocessors, a graphics controller circuit (GPU), or any other electronic component capable of processing inputted data according to specific logical instructions. Having the diagnostic control circuit 136 that includes a GPU may be advantageous for computation-intensive operations, such as volume-rendering. Additionally or alternatively, the diagnostic control circuit 136 may execute instructions stored on a tangible and non-transitory computer readable medium (e.g., the memory 140) to perform one or more operations as described herein.

The memory 140 may be used for storing ultrasound data such as vector data, one or more ultrasound images, acquired ultrasound diagnostic signals, firmware or software corresponding to, for example, a graphical user interface, programmed instructions (e.g., for the diagnostic control circuit 136, the beamformer processor 130, the RF processor 132), and/or the like. The memory 140 may be a tangible and non-transitory computer readable medium such as flash memory, RAM, ROM, EEPROM, and/or the like.

The diagnostic control circuit 136 is operably coupled to a display 138 and a user interface 142. The display 138 may include one or more liquid crystal displays (e.g., light emitting diode (LED) backlight), organic light emitting diode (OLED) displays, plasma displays, CRT displays, and/or the like. The display 138 may display patient information, ultrasound images and/or videos, components of a display interface, one or more 2D, 3D or 4D ultrasound images based on the acquired ultrasound data stored in the memory 140, measurements, diagnostics, treatment information, and/or the like received by the display 138 from the diagnostic control circuit 136.

The user interface 142 controls operations of the diagnostic control circuit 136 and is configured to receive inputs from the user. The user interface 142 may include a keyboard, a mouse, a touchpad, one or more physical buttons, and/or the like. Optionally, the display 138 may be a touch screen display, which includes at least a portion of the user interface 142.

For example, a portion of the user interface 142 may correspond to a graphical user interface (GUI) generated by the diagnostic control circuit 136, which is shown on the display. The GUI may include one or more interface components that may be selected, manipulated, and/or activated by the user operating the user interface 142 (e.g., touch screen, keyboard, mouse). The interface components may be presented in varying shapes and colors, such as a graphical or selectable icon, a slide bar, a cursor, and/or the like. Optionally, one or more interface components may include text or symbols, such as a drop-down menu, a toolbar, a menu bar, a title bar, a window (e.g., a pop-up window) and/or the like. Additionally or alternatively, one or more interface components may indicate areas within the GUI for entering or editing information (e.g., patient information, user information, diagnostic information), such as a text box, a text field, and/or the like.

In various embodiments, the interface components may perform various functions when selected, such as measurement functions, editing functions, database access/search functions, diagnostic functions, controlling acquisition settings, and/or system settings for the ultrasound imaging system 100 and performed by the diagnostic control circuit 136. For example, the interface components may correspond to user selections indicative of the treatment location.

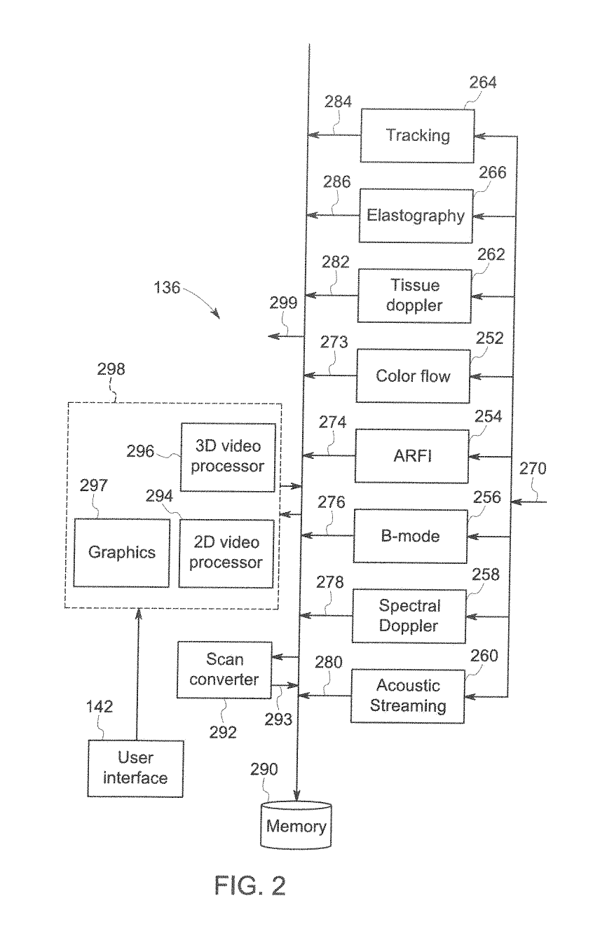

FIG. 2 is an exemplary block diagram of the diagnostic control circuit 136. The diagnostic control circuit 136 is illustrated in FIG. 2 conceptually as a collection of circuits and/or software modules, but may be implemented utilizing any combination of dedicated hardware boards, DSPs, one or more processors, FPGAs, ASICs, a tangible and non-transitory computer readable medium configured to direct one or more processors, and/or the like.

The circuits 252-266 perform mid-processor operations representing one or more operations or modalities of the ultrasound imaging system 100. The diagnostic control circuit 136 may receive ultrasound data 270 (e.g., 3D ultrasound data) in one of several forms. In the embodiment of FIG. 1, the received ultrasound data 270 constitutes IQ data pairs representing the real and imaginary components associated with each data sample of the digitized signals. The IQ data pairs are provided to one or more circuits, for example, a color-flow circuit 252, an acoustic radiation force imaging (ARFI) circuit 254, a B-mode circuit 256, a spectral Doppler circuit 258, an acoustic streaming circuit 260, a tissue Doppler circuit 262, a tracking circuit 264, and an elastography circuit 266 (e.g., shearwave imaging, strain imaging). Other circuits may be included, such as an M-mode circuit, power Doppler circuit, among others. However, embodiments described herein are not limited to processing IQ data pairs. For example, processing may be done with RF data and/or using other methods. Furthermore, data may be processed through multiple circuits.

Each of circuits 252-266 is configured to process the IQ data pairs in a corresponding manner to generate, respectively, color flow data 273, ARFI data 274, B-mode data 276, spectral Doppler data 278, acoustic streaming data 280, tissue Doppler data 282, tracking data 284, electrography data 286 (e.g., strain data, shear-wave data), among others, all of which may be stored in a memory 290 (or the memory 140 shown in FIG. 1) temporarily before subsequent processing. The data 273-286 may be stored, for example, as sets of vector data values, where each set defines an individual ultrasound image frame. The vector data values are generally organized based on the polar coordinate system.

A scan converter circuit 292 accesses and obtains from the memory 290 the vector data values associated with one or more ultrasound image frames and converts the set of vector data values to Cartesian coordinates to create one or more ultrasound image frames 293 formatted for display. The ultrasound image frames 293 created by the scan converter circuit 292 may be provided back to the memory 290 for subsequent processing or may be provided to the memory 140. Once the scan converter circuit 292 creates the ultrasound image frames 293 associated with the data, the image frames may be stored in the memory 290 or communicated over a bus 299 to a database (not shown), the memory 140, and/or to other processors (not shown).

The display circuit 298 accesses and obtains one or more of the image frames from the memory 290 and/or the memory 140 over the bus 299 to display the images onto the display 138. The display circuit 298 receives user input from the user interface 142 selecting one or image frames to be displayed that are stored on memory (e.g., the memory 290) and/or selecting a display layout or configuration for the image frames.

The display circuit 298 may include a 2D video processor circuit 294. The 2D video processor circuit 294 may be used to combine one or more of the frames created from the different types of ultrasound information. Successive frames of images may be stored as a cine loop (4D images) in the memory 290 or memory 140. The cine loop represents a first in, first out circular image buffer to capture image data that is displayed in real-time to the user. The user may freeze the cine loop by entering a freeze command at the user interface 142.

The display circuit 298 may include a 3D processor circuit 296. The 3D processor circuit 296 may access the memory 290 to obtain spatially consecutive groups of ultrasound image frames and to create three-dimensional image representations thereof, such as through volume rendering or surface rendering algorithms as are known. The three-dimensional images may be created utilizing various imaging techniques, such as ray-casting, maximum intensity pixel or voxel projection and the like.

The display circuit 298 may include a graphic circuit 297. The graphic circuit 297 may access the memory 290 to obtain groups of ultrasound image frames that have been stored or that are currently being acquired. The graphic circuit 297 may generate ultrasound images that include the anatomical structures within the ROI.

Additionally or alternatively, during acquisition of the ultrasound data, the graphic circuit 297 may generate a graphical representation, which is displayed on the display 138. The graphical representation may be used to indicate the progress of the therapy or scan performed by the ultrasound imaging system 100. The graphical representations may be generated using a saved graphical image or drawing (e.g., computer graphic generated drawing).

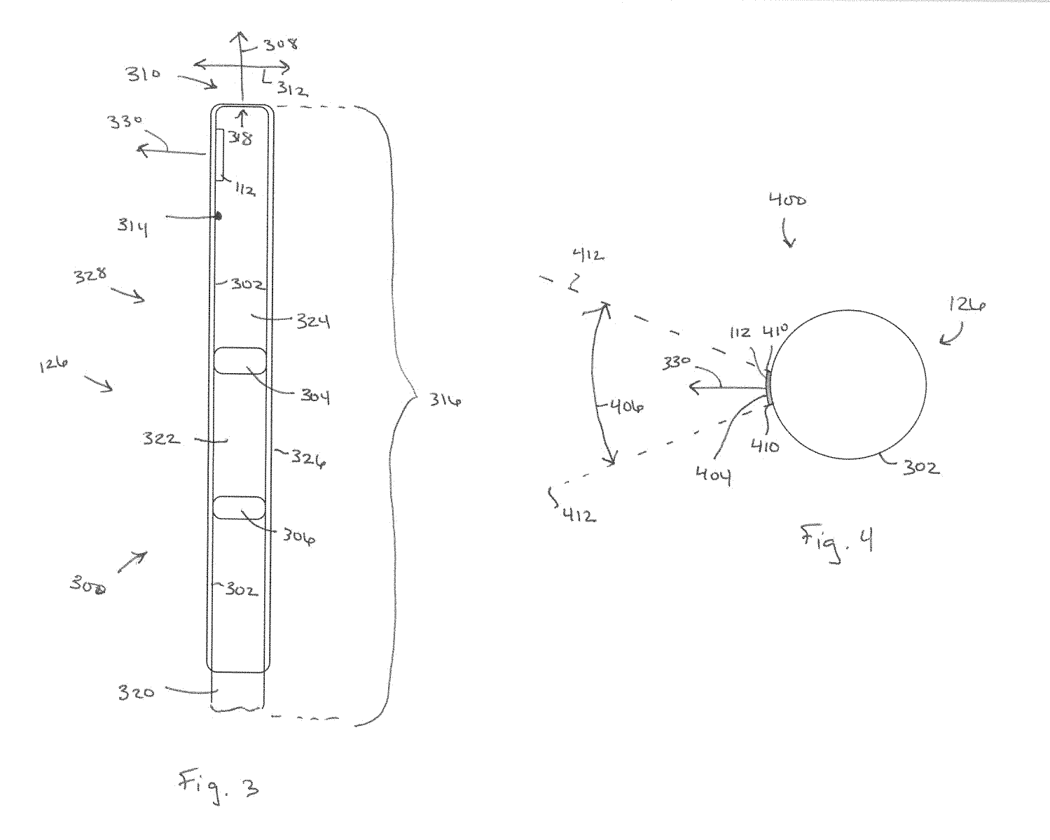

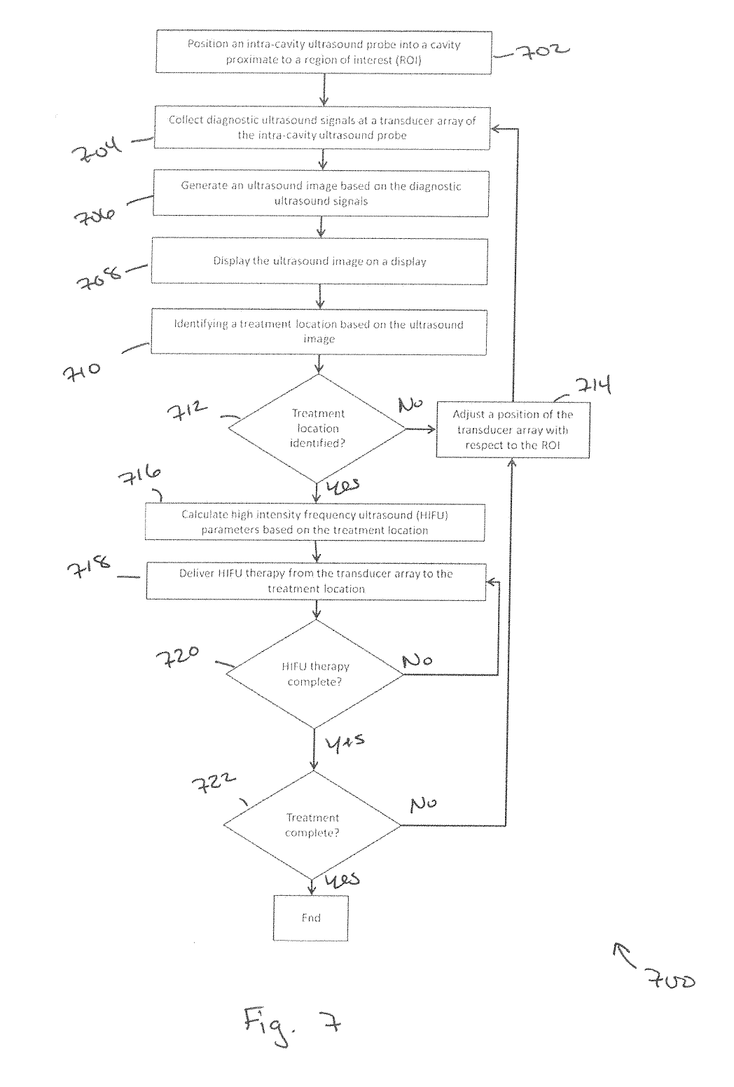

FIG. 3 illustrates a peripheral view 300 of the intra-cavity ultrasound probe 126 of the ultrasound imaging system 100, in accordance with an embodiment. The probe 126 may include a housing 302. The housing 302 may be tubular in shape having a shaft 316. The shaft 316 is elongated along a longitudinal axis 308 terminating at a tip 318. The tip 318 is positioned at a distal end 310 of the probe 126. In various embodiments, the tip 318 may have a planar or flat outer surface aligned along an axis 312 of the distal end 310. Additionally or alternatively, the tip 318 may be angulated (e.g., tip 632 shown in FIG. 6). For example, the tip 318 may not be aligned with the horizontal axis 312. Optionally, during the imaging and HIFU therapies, the housing 302 may be enclosed with a disposable cover or sheet 326. The disposable cover or sheet 326 may be configured to enclose the probe 126 in a sterile surface during use.

The housing 302 may be configured to be inserted into a cavity proximate to the ROI. For example, a diameter of the shaft 316 may be configured to allow for passage through a cervix and into the uterine cavity without cervical dilation. It may be noted in various embodiments, the tip 318 of the shaft 316 may have a reduced diameter relative to the shaft 316. For example, the tip 318 may be configured to have a curved circular edge to reduce a diameter of the tip 318 relative to the shaft 316, such as a diameter ranging from 0.3 to 0.6 mm.

The probe 126 may include segments 320-324 coupled to one another through one or more joints 304-306. An angular position of the joints 304-306 may be managed by an electric motor, pneumatic actuator, and/or the like within the probe 126. The electric motor, pneumatic actuator, and/or the like may be activated and/or controlled via signals generated by the diagnostic control circuit 136. The joints 304-306 may be configured to provide rotational movement of one or more segments 320-324 of the shaft 316 independently with respect to other segment 320-324 of the shaft 316. For example, the joint 306 may provide movement of the segments 322 and 324 independent of the segment 320. In operation, the movement of the segments 322 and 324 by the joint 306 may form an angle of the segment 322 relative to the segment 320. In another example, the joint 304 may provide movement of the segment 324 independent of the segment 320, which may form an angle of the segment 324 relative to the segment 322. It may be noted in other embodiments, the probe 126 may have more than two joints 304-306 or less than two joints 304-306 (e.g., the intra-cavity ultrasound probe 630 in FIG. 6 has no joints).

Optionally, the housing 302 may include one or more apertures 314. The one or more apertures 314 may be positioned proximate to the transducer array 112. The one or more apertures 314 may be configured to produce a suction for attracting and/or removing fluids or liquids (e.g., blood) away from the transducer array 112 and/or the anatomical target. For example, the one or more apertures 314 may be operably coupled to a vacuuming system (not shown) via a tube within the probe 126. The tube may terminate at a reservoir of the vacuuming system. In operation, the one or more apertures 314 may intake fluid proximate to the transducer array 112 and transport the fluid along the tube within the probe 126 to the reservoir.

The housing 302 may include the transducer array 112 located proximate to and/or at the distal end 310 of the housing 302. For example, the transducer array 112 is illustrated positioned along a side 328 at the distal end 318 of the housing 302 such that the transducer array 112 is oriented to face in a lateral direction 330 extending along the horizontal axis 312. Optionally, the transducer array 112 may be configured as a one dimensional array. For example, the transducer elements 124 may be aligned parallel to the longitudinal axis 308 extending along the longitudinal axis 308 along the side of the housing 302. Additionally or alternatively, the transducer array 112 may be a two dimensional array of transducer elements 124.

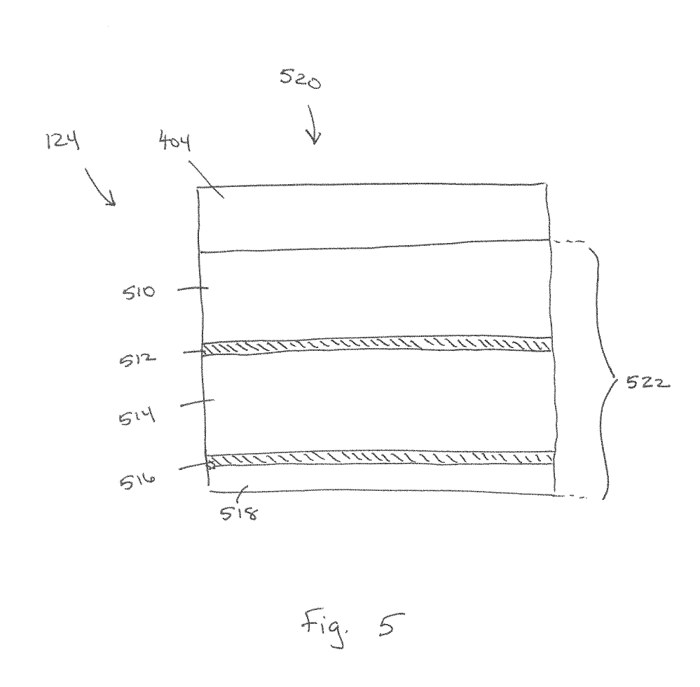

FIG. 4 illustrates a top view 400 of the intra-cavity ultrasound probe 126. The transducer array 112 is positioned directly adjacent to the housing 302. For example, the transducer array 112 is positioned along a surface of the housing 302. The transducer array 112 is shown having an arc shape based on the tubular shape of the housing 302. The arc shape of the transducer array 112 may form a field of view 406 of the transducer array 112. The field of view 406 is a region extending from a face of the transducer array 112. The transducer array 112 is configured to collect diagnostic ultrasound signals and/or transmit HIFU signals within the field of view 406 of the transducer array 112. For example, the field of view 406 may represent an angle through which the transducer array 112 is sensitive to echoes from the ROI, transmit or deliver HIFU signals and/or ultrasound imaging signals, and/or the like. Additionally, extending from the transducer array 112 along the lateral direction 330 is a depth range. For example, the depth range may extend from a proximate end 410 of the field of view 406 on the transducer array 112 to a distal end 412 of the field of view 406. It may be noted that the depth range and the sweep angle arc may be defined within the field of view 406 of the transducer array 112. Optionally, a lens 404 may overlap the transducer array 112. It may be noted in other embodiments (e.g., shown in FIG. 6) the transducer array 112 may be positioned in alternative and/or multiple locations of the housing 302.

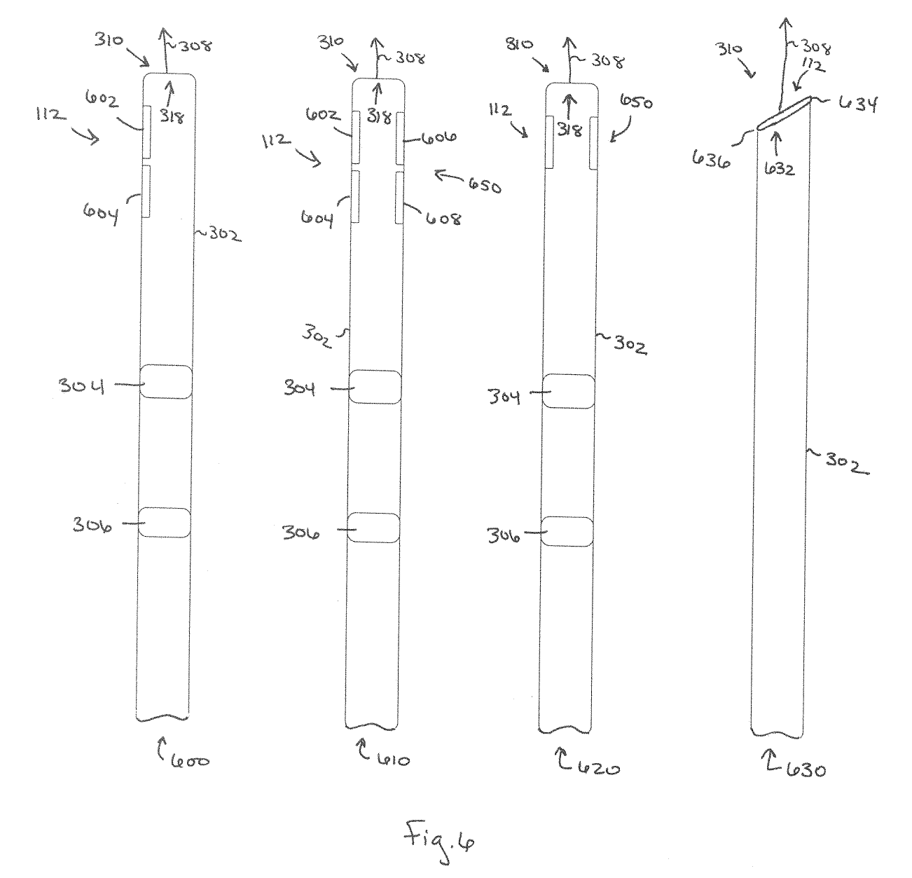

FIG. 5 illustrates a transducer element 124 of the transducer array 112, in accordance with an embodiment. In operation, the transducer element 124 may be combined with a plurality of other transducer elements 124, for example, to form a one dimensional array. It may be noted in other embodiments, the transducer array 112 may be a two dimensional array.

Each of the transducer elements 124 generate ultrasound signals (e.g., acoustic waves) that are directed toward a target. For example, the transducer elements 124 may generate transmit signals directed toward the ROI or the anatomical target. At least a portion of the transmit signals are reflected within the ROI or the anatomical target back toward the transducer element 124 as receive echoes. In another example, the transducer elements 124 may transmit HIFU signals directed toward the treatment location. It may be noted in various embodiments at least one of the transducer elements 124, such as a common transducer element, may be configured to transmit both ultrasound imaging signals and HIFU signals. For example, the diagnostic control circuit 136 may direct at least one common transducer element (e.g., the transducer element 124) of the transducer array 112 to deliver the HIFU therapy to the treatment location, during a therapy session, and to collect the diagnostic ultrasound signals from the ROI, during an imaging session.

Additionally or alternatively, the transducer elements 124 of the transducer array 112 may be grouped into non-therapy transducer elements and non-imaging transducer elements. For example, the non-therapy transducer elements of the transducer array 112 may be configured to transmit ultrasound imaging signals and/or collect the diagnostic ultrasound signals to the ROI when activated by the diagnostic control circuit 136. In various embodiments, when the non-therapy transducer elements are activated by the diagnostic control circuit 136 the non-imaging transducer elements are inactive.

In another example, the non-imaging transducer elements of the transducer array 112 may be configured to deliver the HIFU therapy by generating the HIFU signals to the treatment location when activated by the diagnostic control circuit 136. In various embodiments, when the non-imaging transducer elements are activated by the diagnostic control circuit 136 the non-therapy transducer elements are inactive.

The transducer element 124 may include a lens 404 mounted to an acoustic stack 522. The acoustic stack 522 may include a piezoelectric layer 514 formed from a piezoelectric material (e.g., piezoelectric crystals), or a material that generates an electric charge in response to an applied mechanical force and that generates a mechanical force in response to an applied electric charge. The piezoelectric material may be, for example, lead zirconate titanate (PZT). Alternatively, other piezoelectric materials may be used. While the illustrated transducer element 124 includes only a single piezoelectric layer 514, alternatively a plurality of piezoelectric layers 514 may be provided. For example, the transducer element 124 may include two or more piezoelectric layers 514 stacked on each other.

The piezoelectric layer 514 may be coupled to a ground electrode 512 and a signal electrode 516. The electrodes 512, 516 are electrically conductive bodies, such as layers that include or are formed from one or more metals or metal alloys. The electrodes 512, 516 may be provided as layers that extend over all or substantially all of the footprint of the piezoelectric layer 514, or may be provided as another shape and/or extend over less than all of the footprint of the piezoelectric layer 514. The electrodes 512, 516 may be conductively coupled to probe/SAP electronics, such as the probe/SAP electronics 110 (FIG. 1) by one or more busses, wires, cables, and the like. For example, the probe/SAP electronics 110 control transmission and reception of electronic signals to and from the signal electrode 516. The ground electrode 512 may be conductively coupled to an electric ground reference of the probe/SAP electronics. The ground electrode 512 may convey at least some electric charge generated by the piezoelectric layer 514 to the electric ground reference to avoid interference or crosstalk with the electric charge conveyed to the signal electrode 516.

During imaging guidance or HIFU therapy of the probe/SAP electronics, the signal electrode 516 may receive transmit pulse signals that apply a charge to the signal electrode 516. The applied charge causes the piezoelectric layer 514 to emit ultrasound signals (e.g., acoustic waves), such as the HIFU signals or the ultrasound imaging signals in one or more directions. During the imaging guidance, when the piezoelectric layer 514 receives an acoustic echo, the received acoustic echo may cause mechanical strain in the piezoelectric layer 514, which creates an electric charge in the piezoelectric layer 514. The electric charge is conducted to the signal electrode 516, which conveys the electric charge to the probe/SAP electronics.

Additionally or alternatively, the acoustic stack 522 may be configured to improve efficiency for generating HIFU signals relative to the ultrasound imaging signals. For example, the piezoelectric layer 514 may be configured to have a center frequency or resonance frequency based on the HIFU signals for the HIFU therapy, which is different than the frequency of the ultrasound imaging signals. For example, the piezoelectric layer 514 may have a center frequency ranging from at or about five to seven MHz. Alternatively, the ultrasound imaging signals may occur at or about three MHz. It may be noted in other embodiments, the center frequency may be less than five MHz (e.g., one MHz) or greater than seven MHz.

In another example, the acoustic stack 522 may also include one or more matching layers 510. The matching layers 510 may be configured for a narrow band frequency range relative to the center frequency of the piezoelectric layer 514. For example, the matching layers 510 may be designed for a bandwidth of at or about one MHz. It may be noted in other embodiments the matching layers 510 may have a narrow band than one MHz (e.g., five hundred kHz).

The matching layers 510 are disposed between the lens 404 and the piezoelectric layer 514. For example, the matching layers 510 may be coupled to the lens 404 and the piezoelectric layer 514 on opposing sides of the matching layers 510. The matching layers 510 further have acoustic impedance characteristics between the acoustic impedance characteristics of the piezoelectric layer 514 and the lens 404. For example, the lens 404 may have a relatively low acoustic impedance characteristic while the piezoelectric layer 514 has a relatively large acoustic impedance characteristic. The matching layers 510 may have one or more acoustic impedance characteristics that are greater than the acoustic impedance characteristic of the lens 404 and less than the acoustic impedance characteristic of the piezoelectric layer 514. The intermediate acoustic impedance characteristic(s) of the matching layers 510 can reduce the difference between the acoustic impedance characteristics of the lens 404 and the piezoelectric layer 514. The matching layers 510 can provide a transition region where the mismatch is gradually reduced in order to decrease the reflected acoustic waves.

A backing layer assembly 518 may be disposed below the piezoelectric layer 514. For example, the backing layer assembly 518 may be separated from the piezoelectric layer 514 by the signal electrode 516. Alternatively, the backing layer assembly 518 may at least partially abut the piezoelectric layer 514. The backing layer assembly 516 includes a thermally conductive body (not shown) held within a matrix enclosure. The thermally conductive body may include, or is formed from, one or more materials that conduct thermal energy or heat more than the matrix enclosure. For example, the thermally conductive body may conduct thermal energy away from the piezoelectric layer 514 and other components in the housing (such as other electronic components in a probe head that includes the transducer element 124 and is manipulated by an operator to image a body). In one embodiment, the backing layer assembly 518 may include one or more additional de-matching layers (not shown) disposed between the piezoelectric layer 514 and the thermally conductive body. The de-matching layers can abut the piezoelectric layer 514. The de-matching layers may be relatively thin layers (e.g., less than one wavelength of the acoustic pulses generated by the piezoelectric layer 514). The de-matching layers can have relatively high acoustic impedance characteristics such that the de-matching layers absorb or otherwise reduce the amount or energy of the acoustic pulses that are directed out of the piezoelectric layer 514 toward the thermally conductive body.

In the illustrated embodiment, the lens 404 is a body having a transmission surface 520 through which the ultrasound imaging signals and/or the HIFU signals generated by the piezoelectric layer 514 are emitted. The transmission surface 520 may be a patient engaging surface. For example, the transmission surface 520 may be positioned adjacent or in contact with the anatomical target and/or the treatment location during the imaging guidance and/or HIFU therapy. The lens 404 is mounted to the acoustic stack 522. The lens 404 may be formed from a material having a relatively low acoustic impedance characteristic relative to the piezoelectric layer 514. An acoustic impedance characteristic represents the resistance of a material to the passage of an acoustic wave through the material. For example, the lens 404 may be formed from a silicone rubber. Alternatively or alternatively, the lens 404 may be formed from another material.

FIG. 6 illustrates various intra-cavity ultrasound probes 600, 610, 620, 630, in accordance with various embodiments. The intra-cavity ultrasound probe 600 includes a transducer array 112 that is subdivided into a first set of transducer elements 602 and a second set of transducer elements 604. In operation, the first set of transducer elements 602 may be configured to be activated during the imaging guidance, and the second set of transducer elements 604 are inactive during the imaging guidance. For example, the diagnostic control circuit 136 (FIG. 1) may direct the first set of transducer elements 602 to generate ultrasound imaging signals and to collect the diagnostic ultrasound signals of the ROI, during an imaging session. Additionally or alternatively, the second set of transducer elements 604 may be configured to be activated during the HIFU therapy, and the first set of transducer elements 602 are inactive during the HIFU therapy. For example, the diagnostic control circuit 136 may direct the second set of transducer elements 604 to deliver HIFU therapy by generating HIFU signals to the treatment location, during the therapy session. In various embodiments, the first set of transducer elements 602 may not be activated during the HIFU therapy and the second set of transducer elements 604 may not be activated during the imaging guidance.

The intra-cavity ultrasound probes 610 and 620 may include a second transducer array 650 positioned along opposite sides of the housing 302 such that the transducer array 112 and the second transducer array 650 are oriented to face in a opposite lateral directions relative to the longitudinal axis 308. Optionally, the diagnostic control circuit 136 may select one of the transducer arrays 112, 650 to be activated during the imaging guidance and/or HIFU therapy. In operation, during the imaging guidance and/or HIFU therapy the diagnostic control circuit 136 may determine which of the transducer arrays 112, 650 are activated based on a position of the anatomical target with respect to the intra-cavity ultrasound probe 610, 620. For example, when the diagnostic control circuit 136 determines that the anatomical target is more proximate to the transducer array 112 relative to the second transducer array 650, the diagnostic control circuit 136 may activate the transducer array 112 and the second transducer array 650 is inactive. In another example, the diagnostic control circuit 136 may select one of the transducer arrays 112, 650 based on a user input received by the user interface 142.

Optionally, the second transducer array 650 may be subdivided into a first set of transducer elements 606 and a second set of transducer elements 608. In operation, the first set of transducer elements 606 may be configured to be activated during the imaging guidance. For example, the diagnostic control circuit 136 (FIG. 1) may direct the first set of transducer elements 606 to generate ultrasound imaging signals and to collect the diagnostic ultrasound signals of the ROI, during an imaging session. During the imaging session, the second set if transducer elements 608 may be inactive. Additionally or alternatively, the second set of transducer elements 608 may be configured to be activated during the HIFU therapy. For example, the diagnostic control circuit 136 may direct the second set of transducer elements 608 to deliver HIFU therapy by generating HIFU signals to the treatment location, during the therapy session. During the therapy session, the first set of transducer elements 608 may be inactive. In various embodiments, the first set of transducer elements 606 may not be activated during the HIFU therapy and the second set of transducer elements 608 may not be activated during the imaging guidance.

Additionally or alternatively, the transducer array 112 may be overlaid on a surface area of the tip 632. The tip 632 may be similar to the tip 318. For example, the tip 632 is positioned over the distal end 310 of the intra-cavity ultrasound probe 630. The tip 632 is angulated such that ends 634 and 636 of the tip 632 form a plane of the tip 632 having an angle relative to the longitudinal axis 308. The transducer array 112 may be overlaid on at least a portion of the surface area of the tip 632. For example, the transducer array 112 may be aligned at the angle formed by the tip 632 relative to the longitudinal axis 308. It may be noted in other embodiments, the transducer array 112 may be overlaid on at least a portion of the tip 318. For example, the transducer array 112 may be aligned orthogonal to the longitudinal axis 308 on the tip 318.

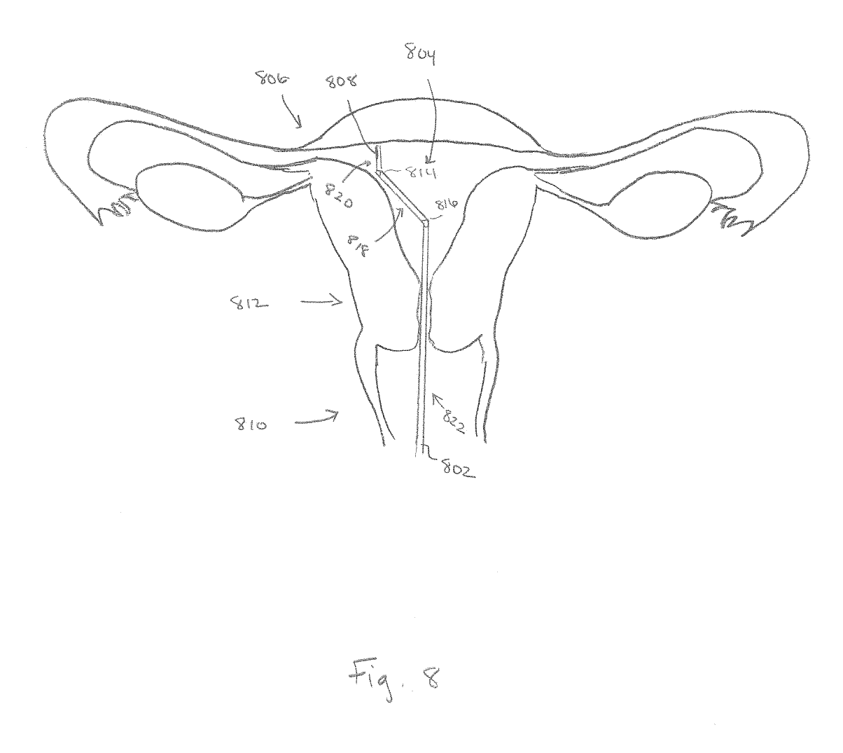

FIG. 7 illustrates a flowchart 700 of a method for delivering HIFU therapy at a treatment location, in accordance with an embodiment. The method 700, for example, may employ structures or aspects of various embodiments (e.g., systems and/or methods) discussed herein. In various embodiments, certain steps (or operations) may be omitted or added, certain steps may be combined, certain steps may be performed simultaneously, certain steps may be performed concurrently, certain steps may be split into multiple steps, certain steps may be performed in a different order, or certain steps or series of steps may be re-performed in an iterative fashion. In various embodiments, portions, aspects, and/or variations of the method 700 may be used as one or more algorithms to direct hardware to perform one or more operations described herein. It may be noted, other methods may be used, in accordance with embodiments herein. Additionally or alternatively, it may be noted that the method 700 may be repeated for subsequent treatments.

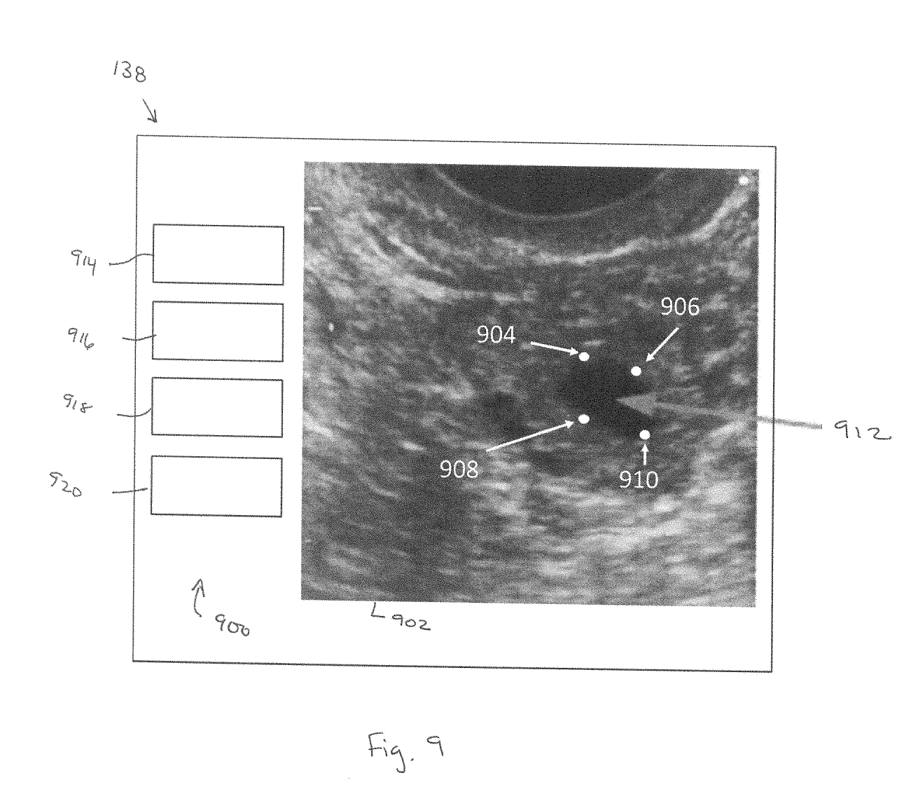

Beginning at 702, an intra-cavity ultrasound probe 802 is positioned into a cavity 804 proximate to a region of interest (ROI) 806. FIG. 8 illustrates the intra-cavity ultrasound probe 802 positioned within the region of interest 806, in accordance with an embodiment. The intra-cavity ultrasound probe 802 may be similar to and/or the same as the intra-cavity ultrasound probe 126, 600, 610, 620, or 630. The cavity 804 shown in FIG. 8 represents a uterine cavity. It may be noted in other embodiments, the intra-cavity ultrasound probe 802 may be positioned within a cavity corresponding to an ear cavity, rectal cavity, and/or the like. The ROI 806 may correspond to one or more areas of the uterine cavity proximate to the fallopian tubes.

The intra-cavity ultrasound probe 802 may be positioned by a clinician (e.g., doctor, nurse, and/or the like). For example, a patient may lie in a supine position with knees bent and a speculum may be inserted into a vaginal cavity 810 to allow visualization of a cervix 812. The intra-cavity ultrasound probe 802 is inserted through the cervix into the cavity 804 (e.g., uterine cavity). Optionally, one or more of joints 814-816 of the intra-cavity ultrasound probe 802 may be activated to position segments 818-820 of the intra-cavity ultrasound probe 802 within the cavity 804. For example, the one or more joints 814-816 may be activated to adjust a distance between a transducer array 808 of the intra-cavity ultrasound probe 802 and the ROI 806. The diagnostic control circuit 136 may activate one or more of the joints 814-816 based on a user input received by the user interface 142. For example, the diagnostic control circuit 136 may activate the joint 816 to rotate the segment 818 with respect to the segment 822. In another example, the diagnostic control circuit 136 may activate the joint 814 to rotate the segment 820 with respect to the segment 818.

At 704, the diagnostic control circuit 136 may direct the transducer array 808 to collect diagnostic ultrasound signals. The transducer array 808 may be similar to and/or the same as the transducer array 112. In operation, the diagnostic control circuit 136 may receive a user input indicative of starting the imaging guidance. During the imaging guidance, the diagnostic control circuit 136 may instruct the transmitter 112 to transmit ultrasound imaging signals. The transmitter 112 may transmit signals to the transmit beamformer 121 and are emitted by the transducer array 808. At least a portion of the ultrasound imaging signals are reflected back from the ROI 806 as echo signals, which are received by the transducer array 808. The receiver 128 may digitize the echo signals to form diagnostic ultrasound signals, which are stored in the memory 140.

At 706, the diagnostic control circuit 136 may generate an ultrasound image based on the diagnostic ultrasound images. For example, the diagnostic control circuit 136 may retrieve the diagnostic ultrasound signals stored in the memory 140 to prepare for the beamformer processor 130. The beamformer processor 130 performs beamforming on the diagnostic ultrasound signals and outputs a radio frequency (RF) signal. The RF signal is then provided to an RF processor 132 that processes the RF signal. The RF processor 132 may create different ultrasound image data types, e.g., B-mode, for multiple scan planes or different scanning patterns. The diagnostic control circuit 136 may be configured to process the acquired ultrasound data (e.g., RF signal data, IQ data pairs, and/or the like) to create one or more ultrasound images.

At 708, the diagnostic control circuit 136 may display the ultrasound image 902 on the display 138. FIG. 9 illustrates the ultrasound image 902 shown on the display 138. The ultrasound image 902 may be shown concurrently with a graphical user interface (GUI) 900. The GUI 900 may include one or more interface components 914-920. One or more of the interface components 914-920 may allow the user to adjust a position of the intra-cavity ultrasound probe 802. For example, the interface component 914 may allow a user to activate the joint 814 to rotate or reposition the segment 820 with respect to the segment 818. Optionally, one or more of the interface components 914-920 may initiate the imaging guidance, the HIFU therapy, and/or the like.

At 710, the diagnostic control circuit 136 may identify a treatment location based on the ultrasound image 902. In operation, the diagnostic control circuit 136 may receive a user input indicative of the treatment location. For example, the user may select or identify one or more user designated points 904-910 on the ultrasound image 902 via the user interface 142. The user designated points 904-910 may be indicative of at least a portion of a boundary of an anatomical target 912 within the ROI 806. The anatomical target 912 may represent an uterotubal junction or corneal junction within the uterine cavity. The user designated points 904-910 may further represent target information received by the diagnostic control circuit 136. For example, the user designated points 904-910 may define a size, shape, depth, and/or the like of the anatomical target 912, which is received by the diagnostic control circuit 136.

Additionally or alternatively, the diagnostic control circuit 136 may identify the treatment location by executing a segmentation algorithm stored in the memory 140. For example, the treatment location may correspond to an area within the ultrasound image 902 with dark or black pixels. The diagnostic control circuit 136 may identify edges of the treatment location based on intensity changes and/or gradients of the vector data values, which form the ultrasound image.

At 712, the diagnostic control circuit 136 may determine whether the treatment location has been identified. Optionally, the diagnostic control circuit 136 may determine that the treatment location has been identified based on a user input. For example, when the designated points 904-910 from the user interface 142 are received by the diagnostic control circuit 136, the diagnostic control circuit 126 may determine that the treatment location has been identified. In another example, the user may select one or more of the interface components 914-920 to indicate the treatment location is not shown in the ultrasound image 902.

If the treatment location is not identified, at 714, a position of the transducer array 808 may be adjust by the diagnostic control circuit 136 with respect to the ROI 806. Optionally, the diagnostic control circuit 136 may activate one or more of the joints 814-816 automatically. For example, the diagnostic control circuit 136 may reposition the segment 820 having the transducer array 808 to include the treatment location in the ultrasound image 902. In another example, the user may select one or more of the interface components 914-920, which activates one or more of the joints 814-816 when the selection is received by the diagnostic control circuit 136.

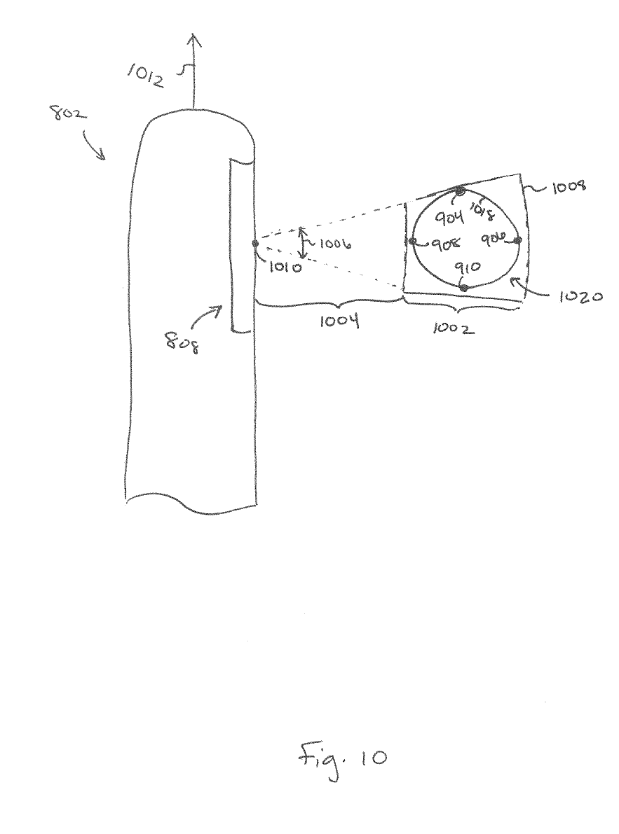

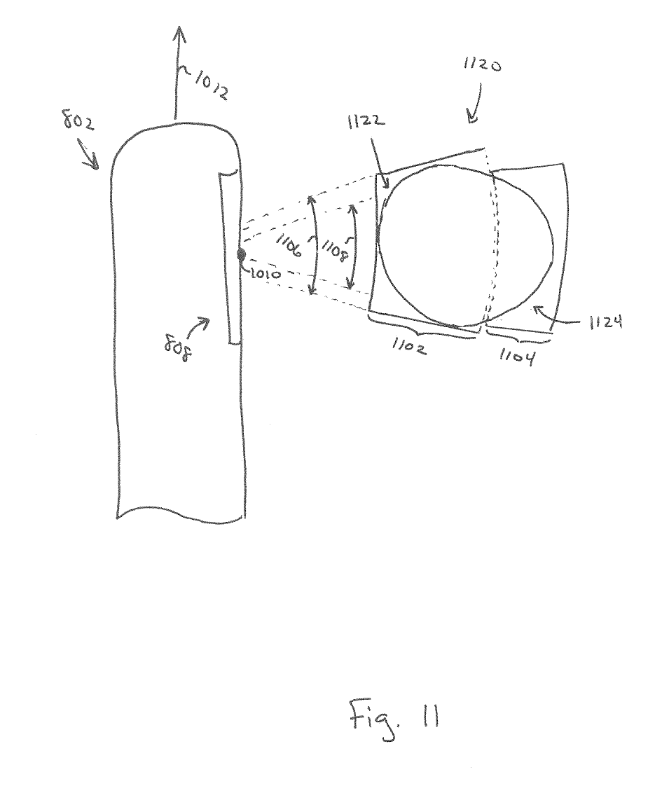

At 716, the diagnostic control circuit 136 may calculate HIFU parameters based on the treatment location. The diagnostic control circuit 136 may receive the target information (e.g., user designated points 904-910) from the memory 140, the user interface 142, and/or the like. Based on the target information, the diagnostic control circuit 136 may determine one or more HIFU parameters (e.g., a depth range, frequency, amplitude or intensity, sweep angle arc, and/or the like). In connection with FIG. 10, the diagnostic control circuit 136 may determine one or more depth ranges 1002 and one or more sweep angle arcs 1006 relative to a reference position 1010 on the transducer array 808.

FIG. 10 illustrates the intra-cavity ultrasound probe 802 and a treatment location 1020. The treatment location 1020 may correspond to an area proximate to and/or within the anatomical target 920 to receive the HIFU therapy defined by or based on the treatment information. The diagnostic control circuit 136 may determine a position of the treatment location 1020 with respect to the reference position 1010 on the transducer array 808. For example, based on the user designated points 904-910 the diagnostic control circuit 136 may determine a distance 1004, orientation, relative position, boundary 1018 (e.g., size, shape), and/or the like of the treatment location 1020 with respect to the reference position 1010. The distance 1004, orientation, relative position, boundary 1018 and/or the like of the treatment location 1020 may be utilized by the diagnostic control circuit 136 to define one or more HIFU parameters. For example, the diagnostic control circuit 136 may determine a depth of the treatment location 1020 based on a distance between the user designated points 906 and 908. In another example, the diagnostic control circuit 136 may determine a length of the treatment location 1020 based on a distance between the user designated points 910 and 904.

Based on a size (e.g., depth, length), shape, and/or the like of the treatment location 1020, the diagnostic control circuit 136 may determine the depth range 1002 and the sweep angle arc 1006 for application of the HIFU therapy to the treatment location 1020.

The depth range 1002 may correspond to a distance or bandwidth from the reference position 1010 on the transducer array 808 for directing the HIFU signals during the HIFU therapy. For example, the depth range 1002 may be a lateral distance range with respect to a longitudinal axis 1012 from the transducer array 808. The depth range 1002 is overlaid on or intersect with at least a portion of the treatment location 1020. The sweep angle arc 1006 may correspond to a steering angle relative to the reference position 1010 on the transducer array 808 for directing the HIFU signals during the HIFU therapy. For example, the sweep angle arc 1006 may be a vertical range aligned with a face of the transducer array 808 (e.g., parallel to the longitudinal axis 1012). In various embodiments, the diagnostic control circuit 136 may define the sweep angle arc 1006 based on an orientation of the target location 1020 relative to the reference position 1010. The sweep angle arc 1006 is overlaid on or intersect with at least a portion of the treatment location 1020.

The depth range 1002 and the sweep angle arc 1006 may be defined by the registration circuit to form a focal point 1008 of the HIFU therapy that includes the treatment location 1020. The focal point 1008 may correspond to a HIFU reception surface area. For example, the focal point 1008 may correspond to a region or surface area of the anatomical target 912 (e.g., the target location) that may be exposed to or interact with the HIFU signals. In various embodiments, the focal point 1008 may be configured by the diagnostic control circuit 136 to be overlaid to and have a size and/or shape similar to and/or approximately the same as the target location 1020.

The diagnostic control circuit 136 may determine a center frequency of the HIFU signals based on the depth range 1002 of the treatment location 1020 relative to the reference position 1010. In operation, the diagnostic control circuit 136 may select a center frequency of the HIFU signals to configure the focal point 1008 by executing one or more algorithms store in the memory 140. In operation, a magnitude of the center frequency adjusts a size of the focal point 1008 at a select distance. For example, a HIFU signal having a center frequency of one MHz may have a focal point 1008 with a diameter of eight to twelve millimeters at a distance of twenty millimeters from the reference position 1010. In another example, a HIFU signal having a center frequency of two MHz may have a focal point 1008 with a diameter of eight to twelve millimeters at a distance of ten millimeters. It may be noted in various embodiments, a length of the distance 1004 may be inversely related to the center frequency of the HIFU signals. For example, HIFU signals delivered to a treatment location at a first distance may have a center frequency greater than HIFU signals delivered to a treatment location at a second distance that is less than the first distance.

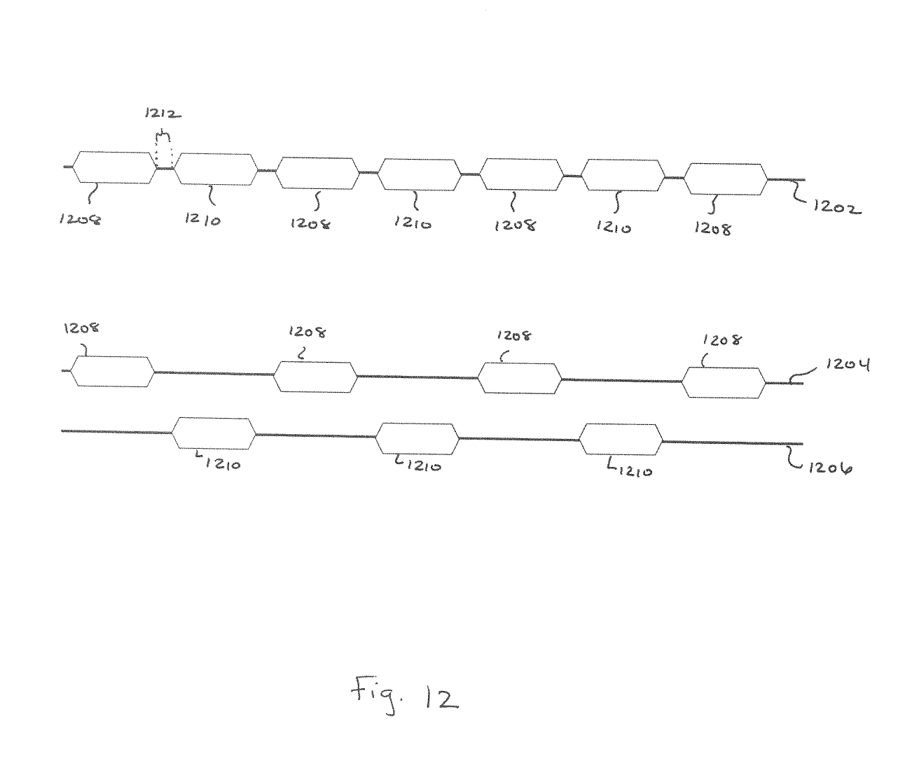

In connection with FIG. 11 the diagnostic control circuit 136 may define a plurality of depth ranges 1102, 1104 and sweep angle arcs 1106, 1108 based on a size of a target location 1120. For example, the diagnostic control circuit 136 may define a plurality of depth ranges 1102, 1104 and sweep angle arcs 1106, 1108 when multiple center frequencies of the HIFU signals are utilized during the HIFU therapy. It may be noted in various embodiments, the diagnostic control circuit 136 may define a plurality of center frequencies, depth ranges and sweep angle arcs based on a plurality of target locations. For example, the diagnostic control circuit 136 may define a first center frequency for treatment locations proximate to the transducer array 808 and a second center frequency of the HIFU signals in connection with treatment locations distal from the transducer array 808.

FIG. 11 illustrates the intra-cavity ultrasound probe 802 and the treatment location 1120. The treatment location 1120 may correspond to an area proximate to and/or within the anatomical target 920 to receive the HIFU therapy defined by or based on the treatment information. A size of the treatment location 1120 may be greater than a size of the treatment location 1020 shown in FIG. 10. Based on a size of treatment locations, the diagnostic control circuit 136 may determine that the target location 1120 may be subdivided into a plurality of focal points 1122, 1124. For example, the diagnostic control circuit 136 may determine that a focus point formed by a single center frequency of the HIFU signals may not include the size of the treatment location 1120 within a set non-zero threshold.

The diagnostic control circuit 136 may execute one or more algorithms stored in the memory 140 to determine a number of focal points to define for the target location 1120. For example, the diagnostic control circuit 136 may calculate a plurality of candidate depth ranges and sweep angle arcs for the target location. The diagnostic control circuit 136 may select a subset of the candidate depth ranges and sweep angle arcs that define a minimal number of focal points and cover the target location 1120. In connection with FIG. 11, the diagnostic control circuit 136 may select two focal points 1122, 1124 having different depth ranges 1102, 1104 and different sweep angle arcs 1106, 1108. For example, the focal point 1122 may have the depth range 1102 and the sweep angle arc 1106, and the focal point 1124 may have the depth range 1104 and the sweep angle arc 1108. It may be noted that although the focal points 1122, 1124 do not have similar depth ranges 1102, 1104 and/or sweep angle arcs 1106, 1108, in various embodiments the diagnostic control circuit 136 may define at least two focal points having a similar or same depth range and/or sweep angle arc.

Additionally or alternatively, the diagnostic control circuit 136 may define different center frequencies of the HIFU signals for each of the focal points 1122, 1124 based on the different depth ranges 1102, 1104. For example, the diagnostic control circuit 136 may determine a first center frequency of the HIFU signals delivered to the focal point 1122 during a first HIFU therapy and a second center frequency for the HIFU signals delivered to the focal point 1124 during the second HIFU therapy.

At 718, the diagnostic control circuit 136 may deliver the HIFU therapy from the transducer array 808 to the treatment location 1020. For example, in connection with FIG. 10, the diagnostic control circuit 136 (FIG. 1) may be configured to direct one or more of the transducer elements 124 in the transducer array 122 transmitter 112 to deliver the HIFU signals defined by the HIFU parameters for the focal point 1008. The diagnostic control circuit 136 may additionally transmit and/or instruct the transmit beamformer 121 to define the depth range 1002 and sweep angle arc 1006 during the HIFU therapy.

In another example, in connection with FIG. 10, the diagnostic control circuit 136 (FIG. 1) may alternate delivery of multiple HIFU therapies between focal points 1122, 1124. The focal points 1122 and the focal points 1124 may have first and second HIFU therapies based on the different HIFU parameters defined by the diagnostic control circuit 136 for each focal point 1122, 1124. For example, the first HIFU therapy may correspond to a first center frequency, depth range 1102, and sweep angle arc 1106. Alternatively, the second HIFU therapy may correspond to a second center frequency, depth range 1104, and sweep angle arc 1108. During application of the HIFU therapy at 718, the diagnostic control circuit 136 may alternate between the first and second HIFU therapies. For example, the diagnostic control circuit 136 utilizing the first center frequency to deliver the first HIFU therapy in connection with the focal point 1122 proximate to the transducer array 808, and the diagnostic control circuit 136 utilizing the second center frequency to deliver the second HIFU therapy in connection with the focal point 1124 distal from the transducer array 808.

Additionally or alternatively, the diagnostic control circuit 136 may deliver multiple HIFU therapies successively. For example, the diagnostic control circuit 136 may deliver the second HIFU therapy when the first HIFU therapy is completed.

Optionally, the diagnostic control circuit 136 may update the ultrasound image shown on the display 138 during delivery of the HIFU therapy. For example, the transducer array 808 may switch to an imaging session during the therapy session to collect the diagnostic ultrasound signals to create the updated ultrasound images (e.g., frames).

FIG. 12 illustrates a timing diagram 1202-1206 of activation of transducer elements of the transducer array 808 during a therapy session. It may be noted that the transducer elements of the transducer array 808 may be similar to and/or the same as the transducer elements 124 of the transducer array 112. Each of the timing diagrams 1202-1206 may represent activation of one or more transducer elements. When activated, the transducer elements transmit ultrasound signals (e.g., ultrasound imaging signals, HIFU signals) and/or collect diagnostic ultrasound signals. Optionally, each of the timing diagrams 1202-1206 may correspond to different sets of transducer elements within the transducer array 808. It may be noted in various embodiments, only the timing diagram 1202 or the timing diagrams 1204-1206 may represent the transducer elements of the transducer array. The timing diagrams 1202-1206 illustrate when the corresponding transducer elements are active for an imaging sessions (e.g., transmitting imaging signals, collecting diagnostic ultrasound signals) during the therapy session. It may be noted that the imaging sessions are interposed between portions of the therapy session.

For example, the timing diagrams 1202-1206 show a first series of activation periods 1208 and a second series of activation periods 1210. The first series of activation periods 1208 may represent the collection of diagnostic ultrasound signals corresponding to an imaging session. The second series of activation periods 1210 may represent the delivery of the HIFU signals by the transducer array 808

The timing diagram 1202 may represent activation of at least one common transducer element of the transducer array 808 during the therapy session. During the therapy session, the one or more common transducer elements are activated to collect the diagnostic ultrasound signals and deliver the HIFU therapy. For example, the one or more common transducer elements are active for the first and second series of activation periods 1208, 1210. In operation, during the first series of activation periods 1208 the diagnostic control circuit 136 may direct the one or more common transducer elements to transmit ultrasound imaging signals and to collect the diagnostic ultrasound signals of the ROI 806 (FIG. 8). In another example, during the second series of activation periods 1210 the diagnostic control circuit 136 may direct the one or more common transducer elements to deliver the HIFU therapy (e.g., transmitting the HIFU signals) to the treatment location 1020. Optionally, between the first and second series of activation periods 1208 and 1210 is an intermediate period 1212. During the intermediate period 1212, the one or more common transducer elements may not be active to allow the piezoelectric layer (e.g., the piezoelectric layer 514) to cool or dissipate heat between activation periods 1208-1210. The intermediate period 1212 may be at or about one millisecond in length. It may be noted in other embodiments the intermediate period 1212 may be longer than one millisecond.

The timing diagrams 1204 may represent a first transducer element and the timing diagram 1206 may represent a second transducer element. In operation, the first transducer element is active during the first series of activation periods 1208, and the second transducer element is inactive during the first series of activation periods 1208. Alternatively, the second transducer element is active during the second series of activation periods 1208, and the first transducer element is inactive during the second series of activations periods 1208. For example, during the first series of activation periods 1208 the diagnostic control circuit 136 may direct the first transducer element to deliver the ultrasound imaging signals and collect the diagnostic ultrasound signals of the ROI 806 (FIG. 8). In another example, during the second series of activation periods 1210 the diagnostic control circuit 136 may direct the second transducer element to deliver the HIFU therapy (e.g., transmitting the HIFU signals) to the treatment location 1020.

At 720, the diagnostic control circuit 136 may determine whether the HIFU therapy is complete. For example, the diagnostic control circuit 136 may measure an elasticity of the treatment location 1020 (FIG. 10). The elasticity of the treatment location 1020 may indicate a formation of scar tissue and/or an occlusion within the ROI 806 and/or the treatment location 1020. For example, the diagnostic control circuit 136 may generate overlay elasticity information on the ultrasound image based on elastography information acquired by the transducer array. The diagnostic control circuit 136 may instruct the transducer array 808 to generate a shearwave directed towards the treatment location 1020 during one or more of the activation periods 1208 to acquire the elastography information of the treatment location 1020. In another example, the diagnostic control circuit 136 may measure temperature information of the treatment location 1020 by tracking tissue expansion. For example, the diagnostic control circuit 136 may calculate temperature information based on distance between echo signals during the activation period 1208 using speckle tracking and/or correlation analysis of the segment of the ultrasound line. In another example, the diagnostic control circuit 136 may receive a user input from the user interface 142 (e.g., selection of one or more interface components 914-920). The user input may be indicative of completion of the HIFU therapy.

If the diagnostic control circuit 136 determine that the HIFU therapy is complete, at 722, the diagnostic control circuit 136 may determine whether the treatment is complete. For example, the diagnostic control circuit 136 may receive a user input from the user interface 142 (e.g., selection of one or more interface components 914-920) indicative of completion of the treatment.

Additionally or alternatively, the diagnostic control circuit 136 may receive a user input from the user interface 142 indicative of returning to an imaging session. Based on the imaging session instruction, the diagnostic control circuit 136 may determine that the treatment is not complete and an alternative treatment location may be selected. For example, the alternative treatment location may correspond to an alternative anatomical target such as the opposing uterotubal junction or corneal junction within the cavity 804. The user may adjust a position of the transducer array 808 with respect to the ROI 806 at 714 to position the transducer array 808 to proximate to the alternative treatment location. In another example, the user may instruct the diagnostic control circuit 136 to activate one or more of the joints 814-816 to reposition the transducer array 808.

In an embodiment a system (e.g., an intra-cavity ultrasound imaging and therapy system) is provided. The system includes an intra-cavity ultrasound probe including a housing configure to be inserted into a cavity proximate to a region of interest (ROI). The housing includes a transducer array located proximate to a distal end of the housing. The system also includes a diagnostic control circuit configured to direct the transducer array to collect diagnostic ultrasound signals from the ROI. The diagnostic control circuit is configured to generate an ultrasound image based on the diagnostic ultrasound signals. The diagnostic control circuit is further configured to direct the transducer array to deliver a high intensity focused ultrasound (HIFU) therapy at a treatment location based on target information derived from the ultrasound image.

Optionally, the transducer array includes transducer elements. The diagnostic control circuit may direct at least one common transducer element to deliver the HIFU therapy to the treatment location, during a therapy session, and to collect the diagnostic ultrasound signals from the ROI, during an imaging session.

Optionally, the probe may include an acoustic stack coupled to the transducer array. The acoustic stack tuned to a select center frequency and bandwidth corresponding to the HIFU therapy.

Optionally, the transducer array includes at least first and second transducer elements. The diagnostic control circuit may direct the first transducer element to collect the diagnostic ultrasound signals of the ROI, during an imaging session. The diagnostic control circuit may direct the second transducer element to deliver the HIFU therapy to the treatment location, during a therapy session.

Optionally, the housing is tubular in shape and elongated along a longitudinal axis. The transducer array may be positioned along a side of the housing such that the transducer array is oriented to face in a lateral direction relative to the longitudinal axis.

Optionally, the system includes a display to display the ultrasound image and a user interface to receive a user input indicative of the treatment location. The diagnostic control circuit may utilize the user input as the target information derived from the ultrasound image to designate the treatment location. Additionally or alternatively, the user input may represent user designated points indicative of at least a portion of a boundary of an anatomical target within the ROI.

Optionally, the diagnostic control circuit defines first and second HIFU therapies having different first and second center frequencies. The diagnostic control circuit may utilize the first center frequency to deliver the first HIFU therapy in connection with treatment locations proximate to the transducer array. The diagnostic control circuit may utilize the second center frequency to deliver the second HIFU therapy in connection with treatment locations distal from the transducer array.

Optionally, the diagnostic control circuit is configured to define at least one of a depth range or a sweep angle arc over which the HIFU therapy is delivered based on the target information.

Optionally, the intra-cavity ultrasound probe includes a plurality of joints. The plurality of joints configured to adjust a distance between the transducer array and the ROI.

Optionally, the diagnostic control circuit is configured to direct only a subset of the transducer elements in the transducer array to deliver the HIFU therapy with a non-therapy subset of the transducer elements remaining inactive during the HIFU therapy.

Optionally, the housing is elongated along a longitudinal axis, the transducer array includes first and second transducer arrays positioned along opposite sides of the housing such that the first and second transducer arrays are oriented to face in opposite lateral directions relative to the longitudinal axis.

In another embodiment a method (e.g., for generating an occlusion by delivering high intensity frequency ultrasound (HIFU) therapy) is provided. The method includes positioning an intra-cavity ultrasound probe into a cavity proximate to a region of interest (ROI). The intra-cavity ultrasound probe includes a housing. The housing includes a transducer array located at a distal end of the housing. The method further collecting diagnostic ultrasound signals at the transducer array from the ROI, and identifying a treatment location based on the diagnostic ultrasound signals. The method further includes delivering high intensity frequency ultrasound (HIFU) therapy from the transducer array to the treatment location.

Optionally, the transducer array includes transducer elements such that the delivering and the collecting operations utilize at least one common transducer element.

Optionally, the collecting of the diagnostic ultrasound signals occurs during an imaging session and the delivering of the HIFU therapy occurs during a therapy session. The imaging session may be interposed between portions of the therapy session.

Optionally, the method includes generating an ultrasound image based on the diagnostic ultrasound signals. The identifying operation may further be based on the ultrasound image. Additionally or alternatively, the method may include displaying the ultrasound image on a display, and receiving a user input indicative of the treatment location from a user interface.

Optionally, the method includes calculating a depth range and a sweep angle arc relative to a reference position on the transducer array based on the target location.

Optionally, the transducer array includes non-therapy transducer element and non-imaging transducer element. The collecting operation may occur at the non-therapy transducer element and the delivering operation may occur at the non-imaging transducer element.

In another embodiment a system (e.g., an intra-cavity ultrasound imaging and therapy system) is provided. The system includes an intra-cavity ultrasound probe including a housing configure to be inserted into a cavity proximate to a region of interest (ROI). The housing includes a transducer array located at a distal end of the housing. The system also includes a diagnostic control circuit configured to direct the transducer array to collect diagnostic ultrasound signals from the ROI. The diagnostic control circuit configured to generate an ultrasound image based on the diagnostic ultrasound signals. The system also includes a display to display the ultrasound image, and a user interface to receive a user input indicative of the treatment location. The diagnostic control circuit is configured to direct the transducer array to deliver a high intensity focused ultrasound (HIFU) therapy at the treatment location.

It may be noted that the various embodiments may be implemented in hardware, software or a combination thereof. The various embodiments and/or components, for example, the modules, or components and controllers therein, also may be implemented as part of one or more computers or processors. The computer or processor may include a computing device, an input device, a display unit and an interface, for example, for accessing the Internet. The computer or processor may include a microprocessor. The microprocessor may be connected to a communication bus. The computer or processor may also include a memory. The memory may include Random Access Memory (RAM) and Read Only Memory (ROM). The computer or processor further may include a storage device, which may be a hard disk drive or a removable storage drive such as a solid-state drive, optical disk drive, and the like. The storage device may also be other similar means for loading computer programs or other instructions into the computer or processor.

As used herein, the term “computer,” “subsystem,” “module,” or “circuit” may include any processor-based or microprocessor-based system including systems using microcontrollers, reduced instruction set computers (RISC), ASICs, logic circuits, and any other circuit or processor capable of executing the functions described herein. The above examples are exemplary only, and are thus not intended to limit in any way the definition and/or meaning of the term “computer”.

The computer or processor executes a set of instructions that are stored in one or more storage elements, in order to process input data. The storage elements may also store data or other information as desired or needed. The storage element may be in the form of an information source or a physical memory element within a processing machine.

The set of instructions may include various commands that instruct the computer or processor as a processing machine to perform specific operations such as the methods and processes of the various embodiments. The set of instructions may be in the form of a software program. The software may be in various forms such as system software or application software and which may be embodied as a tangible and non-transitory computer readable medium. Further, the software may be in the form of a collection of separate programs or modules, a program module within a larger program or a portion of a program module. The software also may include modular programming in the form of object-oriented programming. The processing of input data by the processing machine may be in response to operator commands, or in response to results of previous processing, or in response to a request made by another processing machine.

As used herein, a structure, limitation, or element that is “configured to” perform a task or operation is particularly structurally formed, constructed, or adapted in a manner corresponding to the task or operation. For purposes of clarity and the avoidance of doubt, an object that is merely capable of being modified to perform the task or operation is not “configured to” perform the task or operation as used herein. Instead, the use of “configured to” as used herein denotes structural adaptations or characteristics, and denotes structural requirements of any structure, limitation, or element that is described as being “configured to” perform the task or operation. For example, a controller circuit, processor, or computer that is “configured to” perform a task or operation may be understood as being particularly structured to perform the task or operation (e.g., having one or more programs or instructions stored thereon or used in conjunction therewith tailored or intended to perform the task or operation, and/or having an arrangement of processing circuitry tailored or intended to perform the task or operation). For the purposes of clarity and the avoidance of doubt, a general purpose computer (which may become “configured to” perform the task or operation if appropriately programmed) is not “configured to” perform a task or operation unless or until specifically programmed or structurally modified to perform the task or operation.

As used herein, the terms “software” and “firmware” are interchangeable, and include any computer program stored in memory for execution by a computer, including RAM memory, ROM memory, EPROM memory, EEPROM memory, and non-volatile RAM (NVRAM) memory. The above memory types are exemplary only, and are thus not limiting as to the types of memory usable for storage of a computer program.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the various embodiments without departing from their scope. While the dimensions and types of materials described herein are intended to define the parameters of the various embodiments, they are by no means limiting and are merely exemplary. Many other embodiments will be apparent to those of skill in the art upon reviewing the above description. The scope of the various embodiments should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. §112(f) unless and until such claim limitations expressly use the phrase “means for” followed by a statement of function void of further structure.

This written description uses examples to disclose the various embodiments, including the best mode, and also to enable any person skilled in the art to practice the various embodiments, including making and using any devices or systems and performing any incorporated methods. The patentable scope of the various embodiments is defined by the claims, and may include other examples that occur to those skilled in the art. Such other examples are intended to be within the scope of the claims if the examples have structural elements that do not differ from the literal language of the claims, or the examples include equivalent structural elements with insubstantial differences from the literal language of the claims.

What is claimed is:

1. An intra-cavity ultrasound imaging and therapy system, comprising:
an intra-cavity ultrasound probe including a housing configure to be inserted into a cavity proximate to a region of interest (ROI), the housing including a transducer array located proximate to a distal end of the housing; and
a diagnostic control circuit configured to direct the transducer array to collect diagnostic ultrasound signals from the ROI, the diagnostic control circuit to generate an ultrasound image based on the diagnostic ultrasound signals, the diagnostic control circuit is configured to direct the transducer array to deliver a high intensity focused ultrasound (HIFU) therapy at a treatment location based on target information derived from the ultrasound image.

2. The system of claim 1, wherein the transducer array includes transducer elements, the diagnostic control circuit directing at least one common transducer element to deliver the HIFU therapy to the treatment location, during a therapy session, and to collect the diagnostic ultrasound signals from the ROI, during an imaging session.

3. The system of claim 1, wherein the probe includes an acoustic stack coupled to the transducer array, the acoustic stack tuned to a select center frequency and bandwidth corresponding to the HIFU therapy.

4. The system of claim 1, wherein the transducer array includes at least first and second transducer elements, the diagnostic control circuit to direct the first transducer element to collect the diagnostic ultrasound signals of the ROI, during an imaging session, the diagnostic control circuit to direct the second transducer element to deliver the HIFU therapy to the treatment location, during a therapy session.

5. The system of claim 1, wherein the housing is tubular in shape and elongated along a longitudinal axis, the transducer array positioned along a side of the housing such that the transducer array is oriented to face in a lateral direction relative to the longitudinal axis.

6. The system of claim 1, further comprising a display to display the ultrasound image and a user interface to receive a user input indicative of the treatment location, the diagnostic control circuit to utilize the user input as the target information derived from the ultrasound image to designate the treatment location.

7. The system of claim 6, wherein the user input represents user designated points indicative of at least a portion of a boundary of an anatomical target within the ROI.

8. The system of claim 1, wherein the diagnostic control circuit defines first and second HIFU therapies having different first and second center frequencies, the diagnostic control circuit to utilize the first center frequency to deliver the first HIFU therapy in connection with treatment locations proximate to the transducer array, the diagnostic control circuit to utilize the second center frequency to deliver the second HIFU therapy in connection with treatment locations distal from the transducer array.

9. The system of claim 1, the diagnostic control circuit is configured to define at least one of a depth range or a sweep angle arc over which the HIFU therapy is delivered based on the target information.

10. The system of claim 1, wherein the intra-cavity ultrasound probe includes a plurality of joints, the plurality of joints configured to adjust a distance between the transducer array and the ROI.

11. The system of claim 1, wherein the diagnostic control circuit is configured to direct only a subset of the transducer elements in the transducer array to deliver the HIFU therapy with a non-therapy subset of the transducer elements remaining inactive during the HIFU therapy.

12. The system of claim 1, wherein the housing is elongated along a longitudinal axis, the transducer array includes first and second transducer arrays positioned along opposite sides of the housing such that the first and second transducer arrays are oriented to face in opposite lateral directions relative to the longitudinal axis.

13. A method for generating an occlusion by delivering high intensity frequency ultrasound (HIFU) therapy, the method comprising:
positioning an intra-cavity ultrasound probe into a cavity proximate to a region of interest (ROI), the intra-cavity ultrasound probe including a housing, wherein the housing includes a transducer array located at a distal end of the housing;
collecting diagnostic ultrasound signals at the transducer array from the ROI;
identifying a treatment location based on the diagnostic ultrasound signals; and
delivering high intensity frequency ultrasound (HIFU) therapy from the transducer array to the treatment location.

14. The method of claim 13, wherein the transducer array includes transducer elements, and wherein the delivering and the collecting operations utilize at least one common transducer element.

15. The method of claim 13, wherein the collecting of the diagnostic ultrasound signals occurs during an imaging session and the delivering of the HIFU therapy occurs during a therapy session, the imaging session is interposed between portions of the therapy session.

16. The method of claim 13, further comprising generating an ultrasound image based on the diagnostic ultrasound signals, wherein the identifying operation is further based on the ultrasound image.

17. The method of claim 16, further comprising displaying the ultrasound image on a display; and receiving a user input indicative of the treatment location from a user interface.

18. The method of claim 13, further comprising calculating a depth range and a sweep angle arc relative to a reference position on the transducer array based on the target location.

19. The method of claim 13, wherein the transducer array includes non-therapy transducer element and non-imaging transducer element, the collecting operation occurs at the non-therapy transducer element and the delivering operation occurs at the non-imaging transducer element.

20. An intra-cavity ultrasound imaging and therapy system, comprising:
an intra-cavity ultrasound probe including a housing configure to be inserted into a cavity proximate to a region of interest (ROI), the housing including a transducer array located at a distal end of the housing;
a diagnostic control circuit configured to direct the transducer array to collect diagnostic ultrasound signals from the ROI, the diagnostic control circuit to generate an ultrasound image based on the diagnostic ultrasound signals;
a display to display the ultrasound image; and
a user interface to receive a user input indicative of the treatment location, wherein the diagnostic control circuit is configured to direct the transducer array to deliver a high intensity focused ultrasound (HIFU) therapy at the treatment location.
